(19) United States
(12) Patent Application Publication
(10) Pub. No.: US 2026/0204367 A1
(43) Pub. Date: Jul. 16, 2026

(54) CLINICAL TRIAL DATA VISUALIZATION DEVICE AND METHOD

(71) Applicant: MEDIAIPLUS CO., LTD., Seongnam-si (KR)

(72) Inventors: Yong Jang JO, Seongnam-si (KR); Hong Jo CHAE, Seoul (KR); Ji Hyeon YANG, Seongnam-si (KR); Jae Hyun KIM, Seoul (KR); Ha Jong KIM, Hanam-si (KR); Hyun Min LEE, Hwaseong-si (KR); Joo Eun JEONG, Goyang-si (KR); Ju Hui JUNG, Seoul (KR); Nam Goo SONG, Suwon-si (KR); Ji Hee JUNG, Seoul (KR); Hee Seon PARK, Suwon-si (KR)

(21) Appl. No.: 18/989,343

(22) Filed: Dec. 20, 2024

Related U.S. Application Data
(63) Continuation of application No. PCT/KR2023/016072, filed on Oct. 17, 2023.

(30) Foreign Application Priority Data
Oct. 18, 2022 (KR) 10-2022-0133648

(51) Int. Cl. G16H 10/20; G16H 50/70

(52) U.S. Cl. CPC G16H 10/20; G16H 50/70

(57) ABSTRACT

Disclosed are a clinical trial data visualization device and method for automation of visualization of clinical trial research design schematic diagrams. A clinical trial data visualization device according to one embodiment includes a data extractor which extracts one or more pieces of configuration data for schematic diagram visualization from one or more pieces of clinical trial data stored in a database; and a visualizer which generates clinical trial schematic diagrams on the basis of the one or more pieces of configuration data.

### TECHNICAL FIELD

The present disclosure relates to a clinical trial data visualization device and method for automation of visualization of clinical trial research design schematic diagrams.

### BACKGROUND ART

A clinical trial data search device of the related art provides clinical trial research designs as test listed information. Therefore, users need to directly read a large amount of texts so that it takes a lot of time to understand clinical trial research designs and there are limitations in understanding the contents.

In Korean Unexamined Patent Application Publication No. 10-2020-0005587, features of systems and methods for visualizing a clinical trial site performance and other multi-dimensional (or multi-parameter) datasets are disclosed.

### SUMMARY

### Technical Problem

An object is to provide a clinical trial data visualization device and method for automation of visualization of clinical trial research design schematic diagrams.

### Technical Solution

According to an aspect, a clinical trial data visualization device may include a data extractor which extracts one or more pieces of configuration data for schematic diagram visualization from one or more pieces of clinical trial data stored in a database; and a visualizer which generates clinical trial schematic diagrams on the basis of the one or more pieces of configuration data.

One or more pieces of configuration data may include at least one of population data about a state and the number of clinical trial subjects, intervention data about intervention given to a population to achieve a clinical trial goal, comparison group data about an experimental group and a control group, outcome data about a clinical trial result and an evaluation index, and time data about an evaluation and observation period.

The clinical trial schematic diagram may include a population information node, a population allocation node, and a clinical trial conducting branch and the population information node may be located at the leftmost side, the population allocation node may be located at the right side of the population information node, and the clinical trial conducting branch may be formed starting from a predetermined point of the population allocation node toward the right direction.

The population information node may display at least one of inclusion criteria and exclusion criteria of the clinical trial subjects inside the node and display the number of clinical trial subjects at the outside of the node.

The population allocation node may display a population group allocation method inside the node and display a population masking method at the outside of the node.

The number of clinical trial conducting branches may be determined on the basis of the number of groups of populations and a length of a clinical trial conducting branch may be determined based on a period of a clinical trial duration.

When there are two or more clinical trial conducting branches, the clinical trial conducting branches may be formed to be parallel to each other.

When a second clinical trial duration starts after a first clinical trial duration, a clinical trial conducting branch corresponding to the second clinical trial duration may be formed next to a clinical trial conducting branch corresponding to the first clinical trial duration, and a clinical trial conducting branch of a group of population who receives the same treatment as the first clinical trial duration in the second clinical trial duration, among groups of the population, may be formed in the same direction as a clinical trial conducting branch of the first clinical trial duration.

When a treatment of the second clinical trial duration of a first arm group and a second arm group, among the groups of population, crosses over a treatment of the first clinical trial duration, after crossing over the clinical trial conducting branch of the second arm group, the clinical trial conducting branch of the first arm group may be formed in the same direction as the clinical trial conducting branch of the second arm group on an extending line of the clinical trial conducting branch of the second arm group of the first clinical trial duration. After crossing over the clinical trial conducting branch of the first arm group, the clinical trial conducting branch of the second arm group may be formed in the same direction as the clinical trial conducting branch of the first arm group on an extending line of the clinical trial conducting branch of the first arm group of the first clinical trial duration.

The clinical trial schematic diagram may further include a period marking line formed to be perpendicular to the clinical trial conducting branch at an end point of the clinical trial conducting branch corresponding to a period end point of a predetermined clinical trial duration.

According to an aspect, a clinical trial data visualization method may include extracting one or more pieces of configuration data for schematic diagram visualization from one or more pieces of clinical trial data stored in a database; and generating clinical trial schematic diagrams on the basis of the one or more pieces of configuration data.

### Effects of the Invention

Clinical trial research design information which has been described as texts is automatically expressed as a schematic diagram to improve UX/UI of clinical trial design and research field services.

### BRIEF DESCRIPTION OF DRAWINGS

FIG. 1 is a diagram of a clinical trial data visualization device according to an embodiment.

FIG. 2 is a diagram of a schematic diagram according to an embodiment.

FIGS. 3, 4, 5, 6A and 6B are exemplary views of a schematic diagram according to an example.

FIG. 7 is a flowchart illustrating a clinical trial data visualization method according to an embodiment.

### DETAILED DESCRIPTION OF EXEMPLARY EMBODIMENTS

Hereinafter, an embodiment of the present disclosure will be described in detail with reference to the accompanying drawings. In the description of the present disclosure, a detailed description of known configurations or functions incorporated herein will be omitted when it is determined that the detailed description may make the subject matter of the present disclosure unclear. Further, the terms to be described below are defined considering the functions in the present disclosure and may vary depending on the intention or usual practice of a user or operator. Accordingly, the terms need to be defined based on details throughout this specification.

Hereinafter, embodiments of a clinical trial data visualization device and method will be described in detail with reference to drawings.

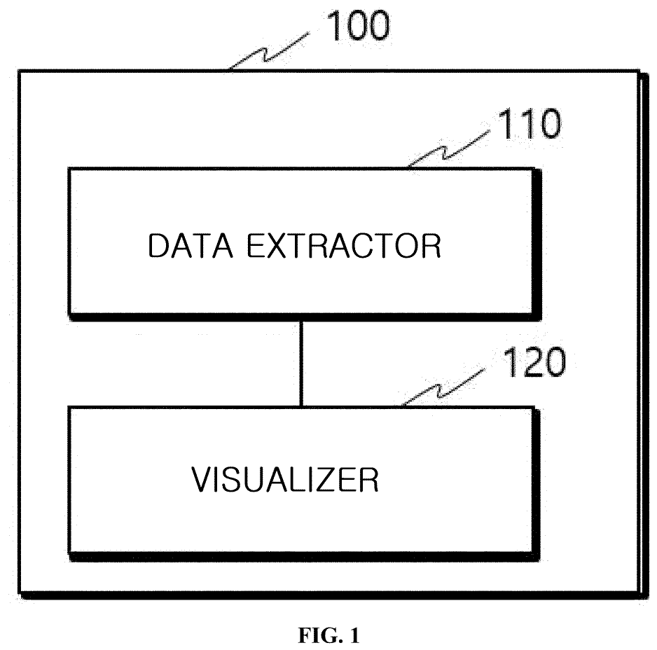

FIG. 1 is a diagram of a clinical trial data visualization device according to an embodiment.

According to an embodiment, the clinical trial data visualization device 100 may include a data extractor 110 which extracts one or more pieces of configuration data for schematic diagram visualization from one or more pieces of clinical trial data stored in a database, and a visualizer 120 which generates clinical trial schematic diagrams on the basis of the one or more pieces of configuration data.

For example, the configuration data may be data about at least one of an indication for the clinical trial, intervention/treatment such as a medicine, a therapy, and a medical device used for the clinical trial, a study type of the clinical trial, the number of clinical trial participants (enrollment), how to allocate a participant group to the clinical trial (allocation), blinding (masking) of a clinical trial investigator, a clinical trial conducting period, start and end dates, the number of clinical trial groups (arm, cohort), a name of medicine used for each arm group, a dose, an administration method, an administration period, an administration frequency, a clinical trial endpoint, an eligibility criteria for clinical trial participants (inclusion criteria, exclusion criteria), a clinical trial phase, a clinical trial progress status, clinical trial sponsors, collaborators, and investigators, and other clinical trial-related information.

According to the embodiment, one or more pieces of configuration data may include at least one of population data about a state and the number of clinical trial subjects, intervention data about intervention given to a population to achieve a clinical trial goal, comparison group data about experimental group and control group, outcome data about a clinical trial result and an evaluation index, and time data about an evaluation and observation period.

According to an example, the clinical trial data visualization device 100 may visualize and provide PICOT (population (patients), intervention, comparison group, outcome, and time) information that a user wants to know. For example, the clinical trial data visualization device 100 may extract PICOT information to generate a clinical trial design schematic diagram from unstructured text data by applying a rule-based and NLP technologies and calls data of a database to automatically generate a schematic diagram on the basis of a visualization rule and may allow a user to directly edit the generated schematic diagram.

According to the embodiment, the data extractor 110 may extract biomedical information for schematic diagram visualization using a natural language processing technology. For example, the data extractor 110 may apply named entity recognition and extract a drug, a dosage, an administration cycle or frequency, an administration duration, and a treatment name as entities. Further, the data extractor 110 may perform semantic role labeling by assigning a dosage, an administration cycle, an administration duration, and a treatment name to a recognized drug.

According to the embodiment, the data extractor 110 may find out a sentence having the same expression (for example, washout, recovery period, run-in period, or a taper period) as a washout period from the unstructured text and extract a time duration. Further, the data extractor 110 may estimate the expression of “period+(washout) period” as a washout period.

According to the embodiment, the data extractor 110 may search the database to perform statistical and calculation processing on the data relationship. For example, as the period, the data extractor 110 may provide a period when study subjects for each arm group actually take the drug through one or more clinical trial durations. However, the entire clinical trial period may be calculated by subtracting a research start date from data of expected or actual completion date data. Further, the data extractor 110 may collect and map paper and academic conference data to extract the number of populations for each arm group and may calculate the patient ratio for each group by comparing it with the total number of clinical trial participants. The data extractor 110 may collect paper and academic conference data and compare it with currently held text data to calculate the observation period until the outcome appears after the end of the clinical trial.

According to an embodiment, the clinical trial schematic diagram may include a population information node, a population allocation node, and a clinical trial conducting branch.

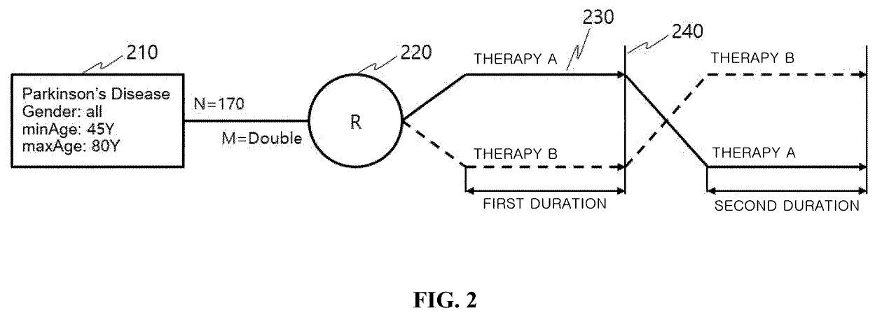

Referring to FIG. 2, the population information node 210 may be located at the leftmost side, the population allocation node 220 may be located at the right side of the population information node 210, and the clinical trial conducting branch 230 may be formed starting from a predetermined point of the population allocation node 220 toward the right direction.

According to an example, the population information node may display at least one of inclusion criteria and exclusion criteria of the clinical trial subjects inside the node and display the number of clinical trial subjects at the outside of the node. For example, the inclusion criteria and exclusion criteria of the clinical trial subject are contents indicating which status of the population (patients or healthy people) is subject to the clinical trial and may include information about which diseases of patients will be included in the clinical trial, which disease will be excluded from the clinical trial, and which age, age group, or gender of people will be included in the clinical trial. The number of clinical trial subjects is the number of people who participate in the clinical trial and may indicate the number of populations or the number of patients.

Referring to FIG. 2, the population information node 210 may display information about the clinical trial subject, “Parkinson's Disease, Gender: all, minAge: 45 Y, maxAge: 80 Y”, thereinside and display the number of clinical trial subjects as “N=170” at the outside.

According to the embodiment, the population allocation node may display a population group allocation method inside the node and display a population masking method at the outside of the node. For example, the group allocation method of the population may include how to assign a drug to an administration group and how to assign a study subject to a comparison group and a control group. For example, the group allocation method of the population may be displayed as “randomized ®” or “nonrandomized (N)”.

According to an example, the masking method is a design method to avoid information bias in the clinical trial and refers to a method of ensuring that no person or department involved in the clinical trial knows which group the population (subject) has been assigned to. For example, the masking method may include single blinding when only the clinical trial subject does not know, double blinding when both the subject and an investigator do not know, quadruple blinding (blinding of a participant, a care provider, an investigator, an outcomes assessor), and no blinding (none, open label).

According to an embodiment, the number of clinical trial conducting branches may be determined based on the number of groups of populations. According to an example, a line indicating an arm group may be expressed as a branch and an arm group type may be distinguished by colors or line types (straight line or dotted line). Further, in the clinical trial conducting branch, a drug (intervention), a dose, a cycle, and a clinical trial period applied to a corresponding arm group may be described as texts for each arm group.

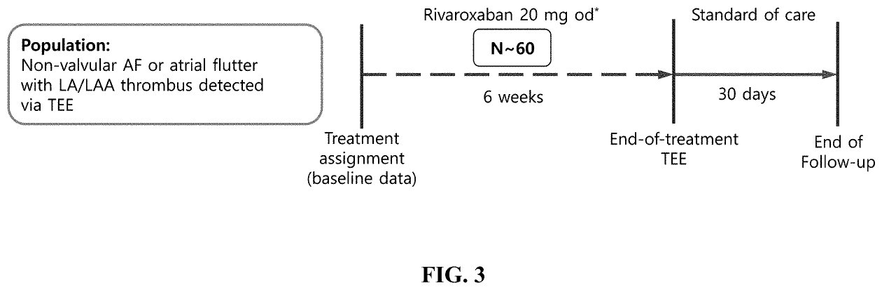

Referring to FIG. 3, the number of population groups is 1, and 20 mg of Rivaroxaban is administrated for first six weeks and a standard of care is followed for 30 days after the end of the treatment.

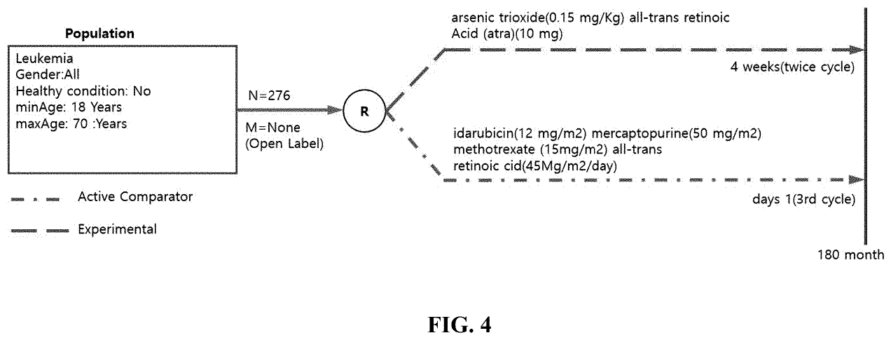

Referring to FIG. 4, the number of population groups is 2 and in the clinical trial “Phase III Trial in Acute Promyelocytic Leukaemia Patients”, the clinical groups of 276 population' ('N=‘76’) with “leukaemia, for all genders, with no healthy condition, aged between 18 and 70 ye'rs' are divided without applying maskin' ('Open La'el') and randomly allocating the subjects (randomized). There are two arm groups including an experimental group in which 0.15 mg/Kg of arsenic trioxide and 10 mg of all-trans retinoic acid (atra) are administrated at twice cycle for four weeks and an active control group (active comparator) in which 12 mg/m2 of idarubicin” is administrated for days 1 (third cycle).

For example, the group of the population may be divided by a medicine (intervention/treatment) used for the clinical trial, a dose of the medicine, an administration method, an administration period, and an administration frequency. For example, the arm group may include an experimental group (corresponding to a drug of interest), an active comparator (mainly corresponding to existing drugs), a placebo comparator (corresponding to a material with no therapeutic effect), a sham comparator (mainly corresponding to actions without therapeutic meaning), and a no-intervention group.

According to an example, the length of a clinical trial conducting branch may be determined based on a period of a clinical trial duration. For example, the period of the clinical trial duration is a treatment period for each group and may indicate a period when patients of each arm group actually take medicines.

According to one embodiment, when the number of clinical trial conducting branches is two or larger, the clinical trial conducting branches may be formed to be parallel to each other. For example, when the clinical trial is designed in parallel, two or more branches may be formed in parallel to each other. For example, the parallel design is a design which is the most commonly adopted to evaluate the efficacy and the safety of new drugs under development and divides the subjects into two or more groups. If there is a placebo or a standard medicine as a control arm, an active comparator is administered to one group and a trial drug is administered to the other group as an experimental group. Each group takes only the same type of medicine during one clinical trial duration from the first administration to the end of administration.

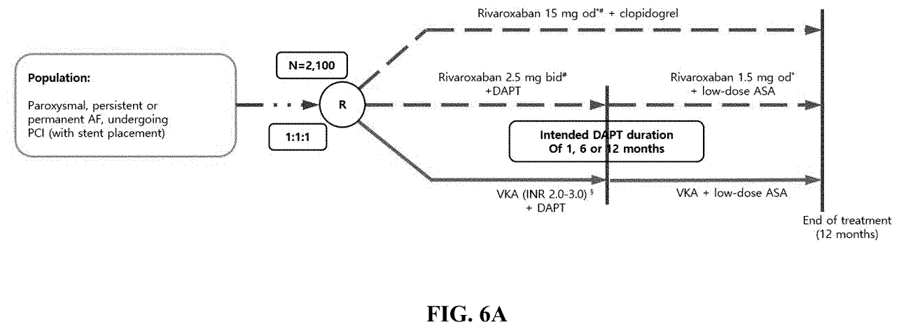

According to the embodiment, if a second clinical trial duration starts after the first clinical trial duration, the clinical trial conducting branch corresponding to the second clinical trial duration may be formed next to the clinical trial conducting branch corresponding to the first clinical trial duration. For example, a clinical trial conducting branch of a group of population which receives the same treatment as the first clinical trial duration, in the second clinical trial duration, among the groups of population, may be formed in the same direction as the clinical trial conducting branch of the first clinical trial duration. Referring to FIG. 6A, the clinical trial duration may be divided with respect to a specific event and when the same drug is used in each duration, the clinical trial conducting branch may be formed continuously in the same direction.

For example, the clinical trial duration refers to a period when the arm group is subject to a predetermined specific treatment, such as a predetermined drug or a predetermined dosage and should be interpreted differently from the clinical trial phase.

According to the embodiment, when a treatment of the second clinical trial duration of a first arm group and a second arm group, among the groups of population, crosses over a treatment of the first clinical trial duration, after crossing over the clinical trial conducting branch of the second arm group, the clinical trial conducting branch of the first arm group may be formed in the same direction as the clinical trial conducting branch of the second arm group on an extending line of the clinical trial conducting branch of the second arm group of the first clinical trial duration. After crossing over the clinical trial conducting branch of the first arm group, the clinical trial conducting branch of the second arm group may be formed in the same direction as the clinical trial conducting branch of the first arm group on an extending line of the clinical trial conducting branch of the first arm group of the first clinical trial duration.

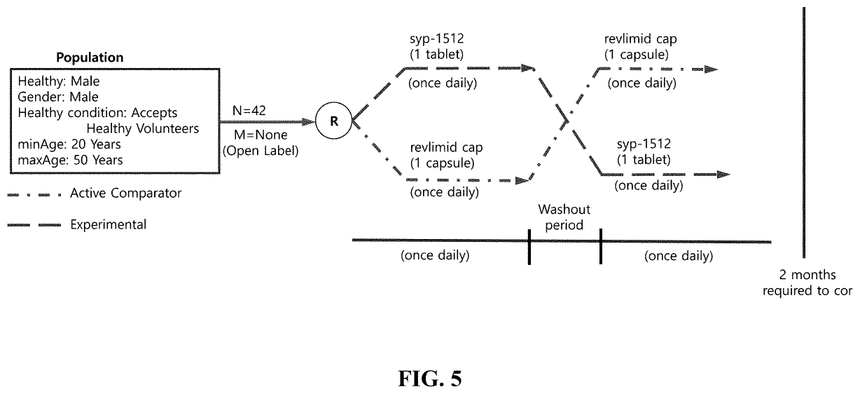

Referring to FIG. 5, it is a schematic diagram of a clinical trial o′ “'Crossover Study to Compare Pharmacokinetic~Healthy Male Volunteers” and a total clinical trial period is two months, a population is a “healthy, male, aged 20 to 50”, the number of clinical trial participants is 42, and masking is not applied (none, open label). A first arm group takes “syp-1512” which is an experimental drug first, and after a washout period, takes a “revlimid” which is an active comparator drug. A second arm group takes “revlimid” first, and then takes “syp-1512”. As illustrated in FIG. 5, when two experimental groups take experimental drugs in a cross-over manner, it is understood that the clinical trial conducting branches indicating two groups cross over each other and then the crossed group is formed in the same direction as a previous clinical trial conducting branch on an extending line of a previous phase of clinical trial conducting branch. Further, the crossing timing may be denoted as a washout period.

According to the embodiment, the clinical trial schematic diagram may further include a period marking line 240 formed to be perpendicular to the clinical trial conducting branch at an end point of the clinical trial conducting branch corresponding to a period end point of a predetermined clinical trial duration. For example, the visualizer 120 may display event information on the schematic diagram to display a specific point of the entire period. For example, the visualizer 120 may display vertical bars indicating specific points such 's 'Study 'nd', ‘Global treatment end d′te’, 'r ‘Efficacy cut-off d′te’, and may display an explanation at the bottom of the bars. An event may be mainly formed with a specific date (a drug administration end point, a group cross-over point) and a study end and may add a period to reach a specific point, such as “6 to 30 months” or “15 to 45 days follow-up”.

According to an example, the visualizer 120 may display clinical trial key test/treatment/therapy keywords extracted from own clinical trial test/treatment/therapy library and context patterns on the schematic diagram. For example, as denoted b′ “'Intended DAPT duration of 1, 6, or 12 months” of FIG. 6A and “Index PCI” of FIG. 6B, when dual antiplatelet therapy (DAPT) or percutaneous coronary intervention (PCI) is performed at the corresponding point, a keyword may be marked at the corresponding point.

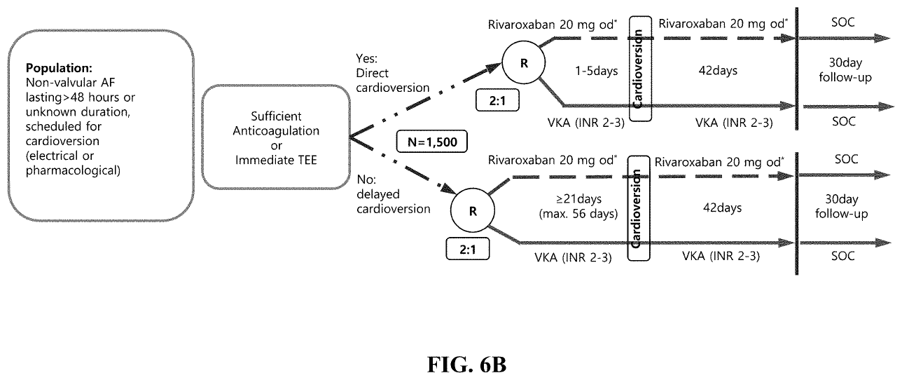

FIG. 7 is a flowchart illustrating a clinical trial data visualization method according to an embodiment.

According to an embodiment, the clinical trial data visualization device may extract one or more pieces of configuration data for schematic diagram visualization from one or more pieces of clinical trial data stored in a database in step 710. For example, one or more pieces of configuration data may include at least one of population data about a state and the number of clinical trial populations, intervention data about intervention given to a population to achieve a clinical trial goal, comparison group data about an experimental group and a control group, outcome data about a clinical trial result and an evaluation index, and time data about an evaluation and observation period.

According to the embodiment, the clinical trial data visualization device may generate clinical trial schematic diagrams on the basis of the one or more pieces of configuration data in step 720.

According to an example, the clinical trial schematic diagram may include a population information node, a population allocation node, and a clinical trial conducting branch. The population information node may be located at the leftmost side, the population allocation node may be located at the right side of the population information node, and the clinical trial conducting branch may be formed starting from a predetermined point of the population allocation node toward the right direction.

Among the embodiments of FIG. 7, embodiments that overlap with the contents described with reference to FIGS. 1 to 6 are omitted.

An aspect of the present disclosure may also be implemented as computer-readable codes written on a computer-readable recording medium. Codes and code segments which implement the program may be easily deducted by a computer programmer in the art. The computer readable recording medium may include all kinds of recording devices in which data, which are capable of being read by a computer system, are stored. Examples of the computer-readable recording media may include ROM, RAM, CD-ROM, magnetic tape, floppy disk, optical disk and the like. Further, the computer readable recording medium may be distributed in computer systems connected through a network to be written and executed with a computer readable code in a distributed manner.

For now, the present disclosure has been described with reference to the preferred embodiments. It is understood to those skilled in the art that the present disclosure may be implemented as a modified form without departing from an essential characteristic of the present disclosure. Accordingly, the scope of the present disclosure is not limited to the above-described embodiment but should be construed to include various embodiments within the scope equivalent to the description of the claims.

### EXPLANATION OF SYMBOLS

100: Clinical trial data visualization device
110: Data extractor
120: Visualizer

What is claimed is:

1. A clinical trial data visualization device, comprising:
a data extractor which generates one or more pieces of configuration data for schematic diagram visualization from one or more pieces of clinical trial data stored in a database; and
a visualizer which generates clinical trial schematic diagrams on the basis of the one or more pieces of configuration data.

2. The clinical trial data visualization device according to claim 1, wherein the one or more pieces of configuration data includes at least one of population data about a state and the number of clinical trial subjects, intervention data about intervention given to the population to achieve a clinical trial goal, comparison group data about an experimental group and a control group, outcome data about a clinical trial result and an evaluation index, and time data about an evaluation and observation period.

3. The clinical trial data visualization device according to claim 1, wherein the clinical trial schematic diagram includes a population information node, a population allocation node, and a clinical trial conducting branch, the population information node is located at the leftmost side, the population allocation node is located at a right side of the population information node, and the clinical trial conducting branch is formed starting from a predetermined point of the population allocation node toward a right direction.

4. The clinical trial data visualization device according to claim 3, wherein the population information node displays at least one of inclusion criteria and exclusion criteria of the clinical trial subjects inside the node and displays the number of clinical trial subjects at an outside of the node.

5. The clinical trial data visualization device according to claim 3, wherein the population allocation node displays a group allocation method of the population inside the node and displays a masking method of the population at an outside of the node.

6. The clinical trial data visualization device according to claim 3, wherein the number of clinical trial conducting branches is determined on the basis of the number of groups of the populations and a length of the clinical trial conducting branches is determined based on a period of a clinical trial duration.

7. The clinical trial data visualization device according to claim 6, wherein when there are two or more clinical trial conducting branches, the clinical trial conducting branches are formed to be parallel to each other.

8. The clinical trial data visualization device according to claim 7, wherein when a second clinical trial duration starts after a first clinical trial duration, a clinical trial conducting branch corresponding to the second clinical trial duration is formed next to a clinical trial conducting branch corresponding to the first clinical trial duration, and a clinical trial conducting branch of a group of population who receives the same treatment as the first clinical trial duration in the second clinical trial duration, among the groups of the population, is formed in the same direction as the clinical trial conducting branch of the first clinical trial duration.

9. The clinical trial data visualization device according to claim 8, wherein when a treatment of the second clinical trial duration of a first arm group and a second arm group, among the groups of population, crosses over a treatment of the first clinical trial duration, after crossing over a clinical trial conducting branch of the second arm group, the clinical trial conducting branch of the first arm group is formed in the same direction as the clinical trial conducting branch of the second arm group on an extending line of the clinical trial conducting branch of the second arm group of the first clinical trial duration, and after crossing over the clinical trial conducting branch of the first arm group, the clinical trial conducting branch of the second arm group is formed in the same direction as the clinical trial conducting branch of the first arm group on an extending line of the clinical trial conducting branch of the first arm group of the first clinical trial duration.

10. The clinical trial data visualization device according to claim 6, wherein the clinical trial schematic diagram further includes a period marking line formed to be perpendicular to the clinical trial conducting branch at an end point of the clinical trial conducting branch corresponding to a period end point of a predetermined clinical trial duration.

11. A clinical trial data visualization method, comprising:
extracting one or more pieces of configuration data for schematic diagram visualization from one or more pieces of clinical trial data stored in a database; and
generating clinical trial schematic diagrams on the basis of the one or more pieces of configuration data.

12. The clinical trial data visualization method according to claim 11, wherein the one or more pieces of configuration data further includes at least one of population data about a state and the number of clinical trial subjects, intervention data about intervention given to the population to achieve a clinical trial goal, comparison group data about an experimental group and a control group, outcome data about a clinical trial result and an evaluation index, and time data about an evaluation and observation period.

13. The clinical trial data visualization method according to claim 11, wherein the clinical trial schematic diagram includes a population information node, a population allocation node, and a clinical trial conducting branch, the population information node is located at the leftmost side, the population allocation node is located at a right side of the population information node, and the clinical trial conducting branch is formed starting from a predetermined point of the population allocation node toward a right direction.

14. The clinical trial data visualization method according to claim 13, wherein the population information node displays at least one of inclusion criteria and exclusion criteria of the clinical trial subjects in the node and displays the number of clinical trial subjects at an outside of the node.

15. The clinical trial data visualization method according to claim 13, wherein the population allocation node displays a group allocation method of the population inside the node and displays a masking method of the population at an outside of the node.

16. The clinical trial data visualization method according to claim 13, wherein the number of clinical trial conducting branches is determined on the basis of the number of groups of populations and a length of the clinical trial conducting branches is determined based on a period of a clinical trial duration.

17. The clinical trial data visualization method according to claim 16, wherein when there are two or more clinical trial conducting branches, the clinical trial conducting branches are formed to be parallel to each other.

18. The clinical trial data visualization method according to claim 17, wherein when a second clinical trial duration starts after a first clinical trial duration, a clinical trial conducting branch corresponding to the second clinical trial duration is formed next to a clinical trial conducting branch corresponding to the first clinical trial duration, and a clinical trial conducting branch of a group of population who receives the same treatment as the first clinical trial duration in the second clinical trial duration, among the groups of the population, is formed in the same direction as the clinical trial conducting branch of the first clinical trial duration.

19. The clinical trial data visualization method according to claim 18, wherein when a treatment of the second clinical trial duration of a first arm group and a second arm group, among the groups of population, crosses over a treatment of the first clinical trial duration, after crossing over the clinical trial conducting branch of the second arm group, the clinical trial conducting branch of the first arm group is formed in the same direction as the clinical trial conducting branch of the second arm group on an extending line of the clinical trial conducting branch of the second arm group of the first clinical trial duration, and after crossing over the clinical trial conducting branch of the first arm group, the clinical trial conducting branch of the second arm group is formed in the same direction as the clinical trial conducting branch of the first arm group on an extending line of the clinical trial conducting branch of the first arm group of the first clinical trial duration.

20. The clinical trial data visualization method according to claim 16, wherein the clinical trial schematic diagram further includes a period marking line formed to be perpendicular to the clinical trial conducting branch at an end point of the clinical trial conducting branch corresponding to a period end point of a predetermined clinical trial duration.
